(19) United States
(12) Patent Application Publication
(10) Pub. No.: US 2018/0325510 A1
(43) Pub. Date: Nov. 15, 2018

(54) BIORESORABLE FIXATION NAIL

(71) Applicant: Syntellix AG, Hannover (DE)

(72) Inventor: Jan Marten Seitz, Hannover (DE)

(21) Appl. No.: 15/777,131

(22) PCT Filed: Nov. 23, 2016
(86) PCT No.: PCT/EP2016/078531

§ 371 Date: May 17, 2018

(30) Foreign Application Priority Data
Nov. 26, 2015 (DE) 10 2015 120 514.9

(51) Int. Cl. A61B 17/064; A61C 8/00; A61B 17/84; A61F 2/30; A61L 31/02; A61L 31/14

(52) U.S. Cl. CPC A61B 17/0642; A61C 8/0028; A61B 17/846; A61B 2017/00964; A61L 31/022; A61L 31/148; A61F 2/30749

(57) ABSTRACT

The invention relates to a fixation nail for temporarily securing transplantable tissue, bone parts, and bone replacement substances using a cover or for securing natural or artificial bone replacement parts to bones. The fixation nail has a round flat nail head with a diameter of 0.50 to 6.0 mm, and the thickness of the nail head is 0.10 to 2.0 mm. The fixation nail has a nail pin which is formed with a sharp tip, and the pin has a length between 0.5 times to 2 times the diameter of the nail head and a thickness between 0.15 times to 0.5 times the diameter of the nail head. The fixation nail consists of a biocompatible, biocorrodable magnesium alloy which is composed of at least 90 wt. % metal magnesium and contains less than 0.1 wt. % aluminum, less than 0.1 wt. % copper, less than 0.1 wt. % iron, and less than 0.1 wt. % nickel as physiologically undesirable impurities.

The present invention relates to a bioresorbable nail for the fixation of membranes which are used to cover bone defects or to attach natural and artificial bone replacement parts to bones.

Tooth loss, periodontitis and the bearing pressure of dental prostheses can cause bone resorption of the jawbone (jawbone resorption) to occur. When implanting a dental prosthesis, it can therefore be necessary to carry out jawbone augmentation in the first instance. In this case, dental implants are a type of supporting pillar anchored in the jawbone, which have the function of an artificial tooth root. If too little bone is available, permanent stable integration of the implant cannot be guaranteed.

If insufficient bone is available, bone augmentation (bone formation) can occur at two stages, i.e. before the actual implantation or also at the same time as the actual implantation of the dental prosthesis. In the case of bone formation, either autologous bone from the same individual is used, which must first be removed from the patient at a suitable location, or foreign material is used. The foreign material can be of natural origin, for example donor bones, or it can be manufactured synthetically. To build and regenerate the jawbone, the defect in the bone is filled with the autologous or foreign material. Synthetic bone substitutes, such as hydroxyapatite and calcium phosphate, are normally used in the form of chippings or granulate. Autologous bone material is normally used in the form of borings.

So that the bone formation material cannot migrate or be washed out and in order to prevent the penetration and ingrowth of soft tissue cells into the filled bone defect site (slowly regenerating hard tissue), this is sealed with a covering membrane (principle of guided bone regeneration, GBR).

Covering membranes are known from the prior art. Well-established covering membranes from the prior art are normally secured to bones by means of nails or screws made of metals such as titanium in order to prevent slipping. Here, mere clamping or sewing of the covering membranes is not sufficient. The period for healing is generally between 12 and 20 weeks.

U.S. Pat. No. 5,957,690 A discloses a covering membrane made of the flexible plastic material polytetrafluorethylene (PTFE).

DE 43 02 709 C1 describes a covering membrane having a reinforcing layer. This membrane is fixed to the jawbone by means of fastening nails.

A covering film moulded from inelastically stress-free and permanently deformable titanium and a corresponding nail for position fixing are disclosed in EP 0 809 979 A1.

Covering membranes which are made of collagen fibres or similar materials and are resorbable by the body are also known.

Non-resorbable fixing means, such as titanium nails or screws, and non-resorbable covering membranes have the disadvantage that the non-resorbable attachment means and covering membranes must be removed again by means of surgical intervention following the insertion of the bone substitute material and healing of the wound before the actual dental implant can be used. This constitutes a significant irritation of the affected region.

In the case of bone defects in other parts of the body, natural or artificial bone replacement parts must be secured to adjoining bones by means of various fixing aids. In this case too, non-resorbable fixing means must normally be surgically removed from the body again as soon as the bone is healed.

The object of the present invention was therefore to provide an alternative attachment means which avoids the known disadvantages from prior art.

The object is solved by a fixing nail according to claim 1.

The fixing nail for temporarily attaching transplantable tissue, bone parts and bone replacement substances by means of covering and for attaching natural and artificial bone replacement parts has a round and flat nail head with a diameter of 0.50 to 6.0 mm, wherein the thickness of the nail head is 0.10 to 2.0 mm and the fixing nail has a nail pin designed with a sharp tip, the length of said nail pin being 0.5 times to 2 times the diameter of the nail head, and the pin thickness of said nail pin being between 0.15 times and 0.5 times the diameter of the nail head, wherein the fixing nail consists of a biocompatible biocorrodable magnesium alloy composed of at least 90 wt. % metallic magnesium and less than 0.1 wt. % aluminium, less than 0.1 wt. % copper, less than 0.1 wt. % iron and less than 0.1 wt. % nickel as physiologically undesired impurities.

If a degradable covering membrane is used in jaw surgery together with the biocompatible and biocorrodable fixing nail, the complete resorption of both the wound-covering membrane and the fixing aid, namely the fixing nails according to the invention, occurs over the course of the wound healing—which generally takes 12 to 20 weeks. In the end there is a reinforced bone surrounded by healthy gums. The surgeon can position the dental implant without first having to remove residues of the membrane, auxiliary aid or titanium pins.

The surgical removal of non-resorbable fixing means is also unnecessary in the case of bone defects in other parts of the body when using the magnesium fixing nail according to the invention.

The advantage over comparable fixing pins or fixing nails made of titanium or stainless steel is that the magnesium nails according to the invention together with the usually likewise degradable covering tissue, such as collagen membrane, are completely resorbed by the body during healing and do not have to be removed by a further surgical intervention before or during the positioning of the actual dental implant. Magnesium also has a bone formation-promoting effect (Switzer, E. N., Resorbierbares metallisches Osteosynthesematerial. Untersuchungen zum Resorptionsverhalten im Meerschweinchen-Modell, Dissertation, Tierarztliche Hochschule Hannover, 2005). It is also known that the magnesium ions released during resorption stimulate bone formation (WO 2015/133963 A).

In a preferred embodiment, the diameter of the nail head is 1.5 to 4.0 mm, the length of the sharpened nail pin is 0.75 to 8.0 mm and the thickness of the nail head is 0.4 to 1.0 mm and the thickness of the nail pin is 0.22 to 2.0 mm. The nail head is thereby preferably designed in the form of a drawing pin.

Furthermore, it is preferred that the diameter of the nail head is preferably 2.5 mm, that the length of the sharpened nail pin is preferably 2.4 mm, that the thickness of the nail head is preferably 0.6 mm and that the thickness of the nail pin is a maximum of 0.8 mm.

The firm fixation of the covering membrane is achieved by means of pushing or hammering the magnesium nail according to the invention into the jawbone.

The fixing nails are used at transplantation sites which are difficult to access, such as in the region of the mandible and maxilla. The surgeon uses a thin pen-shaped applicator in the process which comprises the head of the nail with clamps and facilitates positioning at the appropriate site. The nail is then pressed in using the applicator or hammered into the bone by means of a surgical hammer. For this purpose, the nail pin must have a certain mechanical stability, which is primarily provided by the material thickness of the pin and the chemical composition and manufacturing process of the alloy. The wide head of the nail prevents the nail being driven completely through the covering membrane.

It is advantageous for the anchoring of the nail if one or two barbs are provided in the lower area of the nail pin, approximately below the centre. They increase the strength of the anchoring and make it difficult to remove the nail. In an embodiment of the invention, the nail pin therefore has at least one barb.

It is further preferred that the nail pin has one or two barbs in the region of the lower half of the pin length.

The barb or barbs are preferably produced by the radial twisting of the nail pin by 0.1 to 0.4 mm.

The magnesium nail must guarantee firm fixation of the membrane that covers the inserted bone material or of the transplanted bone piece for a specific period. The speed of resorption of the magnesium nail is primarily defined by the chemical composition of the alloy and its manufacture. Both the mechanical stability of the magnesium pins and their resorption rate are influenced by additives of certain alloy elements, particularly zinc, zirconium and rare earth metals (alloys of yttrium, lanthanum, neodymium, dysprosium and others). The stated minimum and maximum threshold values of the composition of these alloys are selected such that resorption is fully guaranteed within the healing timeframe of the bone transplantation or bone formation.

The physiological safety of the magnesium nails is primarily defined by the content of unavoidable impurities. Maximum quantities of physiological harmful trace elements are therefore stated. Impurities caused by aluminium, iron, nickel and copper are each particularly limited to values below 0.1 wt. % in the magnesium nails according to the invention. In general terms, the actual quantities of impurities are preferably below 0.01 wt. %.

The physiological safety of magnesium as a biocorrodable metal is known from other studies (Schrenk, Sebastian, “Abbauverhalten degradierbarer Magnesiumlegierungen in korperahnlichen Flussigkeiten”, Dissertation, Medizinische Fakultat der Friedrich-Alexander-Universitat Erlangen-Nurnberg 2011).

The fixing nail preferably consists of a biocorrodable magnesium-based alloy, which is composed of magnesium, yttrium, zirconium and other rare earth metals.

According to a further aspect of the present invention, the fixing nail is made of a biocorrodable magnesium alloy composed of at least 80 wt. % metallic magnesium, a zinc proportion of 0.1 to 3.0 wt. %, a zirconium proportion of 0.1 to 3.0 wt. %, a rare earth metals proportion totalling 0.1 to 10.0 wt. % and less than 0.1 wt. % aluminium, less than 0.1 wt. % copper, less than 0.1 wt. % iron and less than 0.1 wt. % nickel as physiologically undesired impurities, wherein the rest is 100 wt. % magnesium.

It is preferred that the yttrium proportion is 1.5 to 5 wt. % of the rare earth metals proportion totalling 0.1 to 10.0 wt. %.

The magnesium-based alloy preferably contains 0.10 to 2.00 wt. % zirconium, 0.01 to 0.80 wt. % zinc, 1.50 to 5.00 wt. % yttrium and 2.50 to 5.00 wt. % of other rare earth metals.

It is preferred in this regard that the magnesium-based alloys in relation to the alloy have a total content of physiologically undesired impurities of the metals iron, copper, nickel and aluminium of less than 0.02 wt. % in each case. In particular, the magnesium-based alloy contains less than 0.01 wt. % aluminium, less than 0.20 wt. % iron, less than 0.20 wt. % manganese and less than 0.02 wt. % in each case of copper and nickel.

Furthermore, it is preferred that the magnesium-based alloy contains less than 0.01 wt. % aluminium, less than 0.20 wt. % zinc, less than 0.15 wt. % manganese, less than 0.20 wt. % lithium, less than 0.01 wt. % silicon, less than 0.01 wt. % iron, less than 0.03 wt. % copper and less than 0.005 wt. % nickel.

In place of the barbs, it may be sufficient for the tip of the nail to be designed in a harpoon-shaped or lanceolate manner.

It is therefore further preferred that the tip of the nail pin is designed in a harpoon-shaped manner.

It is also preferred that the tip of the nail pin is designed in a lanceolate manner.

In a further embodiment, the nail head is designed in a hooded manner.

The nail pin can also be provided with grooves over its entire length or a part of its length particularly in order to improve the initial anchoring into the bone and to prevent any unwanted extraction.

In a further preferred embodiment, the nail pin therefore has at least one, preferably 2 to 5 groove-like constrictions of 0.1 to 0.2 mm in the region of the lower third of the fixing pin.

In an embodiment, the nail pin has at least one bulge of 0.1 to 0.3 mm in the region of the lower third of the fixing pin.

Further advantages arise from using the fixing nail according to the invention for temporary attachment of stabilising tissue during bone augmentations. The use of the fixing nail according to the invention is therefore not restricted to just the field of maxillofacial surgery but can also be used to fix natural or artificial stabilising tissues and natural and artificial bone material to the body's own bones.

In addition, a use of the fixing nail according to the invention is provided for temporary attachment of resorbable and non-resorbable covering membranes for dental implants. This preferably relates to a use for temporary attachment of collagen-containing and bone-forming tissue.

The invention in question will be described in greater deal by way of the following figures.

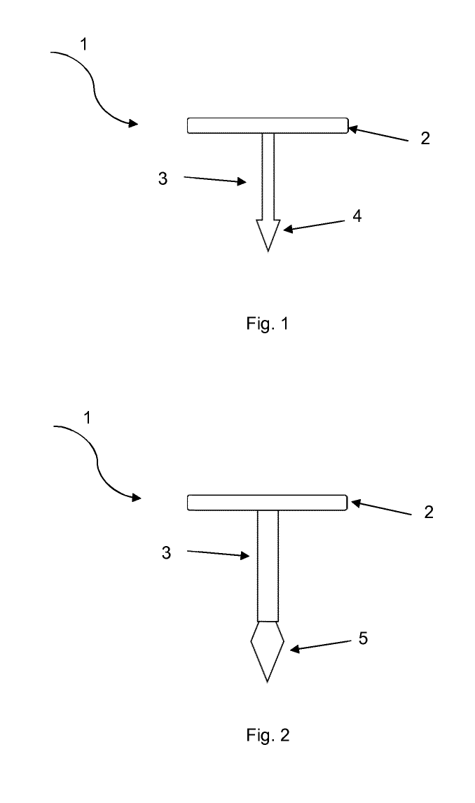

FIG. 1 shows the fixing nail according to the invention having a harpoon-shaped tip of the nail pin.

FIG. 2 shows the fixing nail according to the invention having a lanceolate tip of the nail pin.

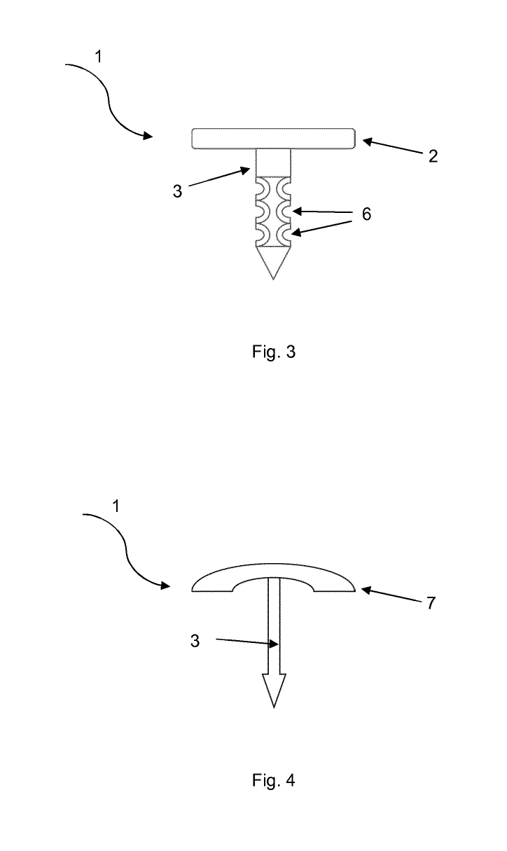

FIG. 3 shows the fixing nail according to the invention having grooves in the nail pin.

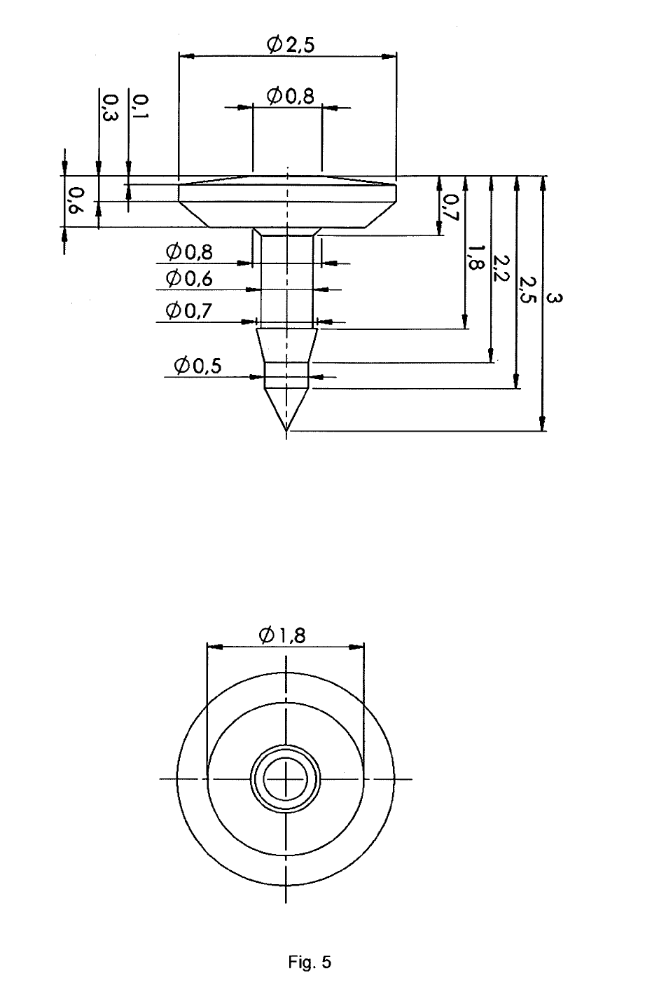

FIG. 4 shows the fixing nail according to the invention having a hooded nail head.

FIG. 5 shows a detailed drawing of a magnesium nail according to the invention for use in dentistry.

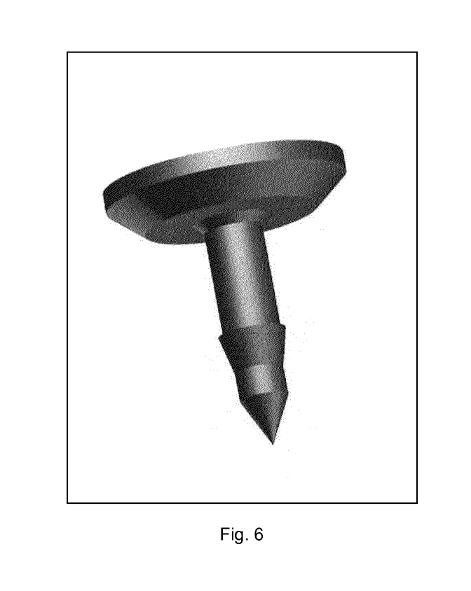

FIG. 6 shows a three-dimensional representation of the magnesium nail according to the invention as per FIG. 5.

FIGS. 1 to 6 each show an embodiment of the fixing nail (1) according to the invention. This consists of a nail head (2) and a nail pin (3). The tip of the nail pin (3) can be harpoon-shaped (4) (FIG. 1) or lanceolate (5) (FIG. 2) in order to ensure stable anchoring in the bone.

The nail pin (3) can also be provided with grooves (6) over the entire length or a part of its length (FIG. 3), particularly in order to improve the initial anchoring in the bone and to prevent any unwanted extraction.

The “tearing” of the nail head through the anchored covering membrane is prevented by the head of the nail, which is broad in relation to the length of the pin. The nail head must also have sufficient mechanical stability and must not bend when the nail is driven in. The nail head can be hooded (7) (FIG. 4).

FIGS. 5 and 6 show a typical embodiment of the magnesium nail according to the invention which has been trialled within the clinic. A particularly suitable embodiment of the invention is shown in FIG. 5. The magnesium nail outlined there in a CAD drawing is characterised by a nail head of 2.5 mm in diameter and a material thickness of 0.7 mm. The nail pin is characterised by a length of 2.4 mm and a material thickness of 0.7 mm. The nail pin has a milled portion of 0.1 mm in the middle to form a barb approximately. The nail head has bevels on the edge which makes it easier to grip the pin with the applicator. FIG. 6 shows the magnesium nail according to the invention in a three-dimensional view. This magnesium nail is particularly suitable for use in bone augmentation in the course of dental implantations.

When using magnesium nails according to the invention, particularly a nail according to FIG. 5 and FIG. 6, all clinical requirements are fulfilled. The physiological safety of magnesium as a biocorrodable metal is known from other studies (Schrenk, Sebastian, “Abbauverhalten degradierbarer Magnesiumlegierungen in korperahnlichen Flussigkeiten”, Dissertation, Medizinische Fakultat der Friedrich-Alexander-Universitat Erlangen-Nurnberg 2011).

### LIST OF REFERENCE SYMBOLS

1. Fixing nail

2. Nail head

3. Nail pin

4. Harpoon-shaped tip of the nail pin

5. Lanceolate tip of the nail pin

6. Grooves

7. Hooded nail head

1. A fixing nail (1) for temporary attachment of transplantable tissue, bone parts and bone replacement substances by means of covering or for attachment of natural and artificial bone replacement parts to bones,
comprising a round and flat nail head (2) having a diameter of 1.5 to 4.0 mm and wherein the thickness of the nail head (2) is 0.4 to 1.0 mm
wherein
the fixing nail (1) has a nail pin (3) designed with a sharp tip, the length of said nail pin being 0.5 times to 2 times the diameter of the nail head, and the pin thickness of said nail pin being between 0.15 times and 0.5 times the diameter of the nail head,
wherein the fixing nail (1) consists of a biocompatible, biocorrodable magnesium alloy composed of at least 90 wt. % metallic magnesium and less than 0.1 wt. % aluminium, less than 0.1 wt. % copper, less than 0.1 wt. % iron and less than 0.1 wt. % nickel as physiologically undesired impurities.

2. The fixing nail (1) according to claim 1, wherein the fixing nail (1) has a round and flat nail head (2) having a diameter of preferably 2.5 mm, and wherein the thickness of the nail head (2) is preferably 0.6 mm and the fixing nail (1) has a nail pin (3) designed with a sharp tip, the length of said nail pin being preferably 2.4 mm, and the pin thickness of said nail pin being a maximum of 0.8 mm.

3. The fixing nail according to claim 1 wherein the nail pin (3) has at least one barb.

4. The fixing nail according to claim 1, wherein the nail pin (3) has one or two barbs in the region of the lower half of the pin length.

5. The fixing nail (1) according to claim 1, the fixing nail (1) consists of a biocorrodable magnesium alloy composed of at least 80 wt. % metallic magnesium, a zinc proportion of 0.1 to 3.0 wt. %, a zirconium proportion of 0.1 to 3.0 wt. %, a rare earth metals proportion totaling 0.1 to 10.0 wt. % and less than 0.1 wt. % aluminium, less than 0.1 wt. % copper, less than 0.1 wt. % iron and less than 0.1 wt. % nickel as physiologically undesired impurities, wherein the rest is 100 wt. % magnesium.

6. The fixing nail (1) according to claim 1 wherein the nail pin (3) has at least one bulge of 0.1 to 0.3 mm in the region of the lower third of the fixing pin.

7. The fixing nail (1) according to claim 1 wherein the nail pin (3) has at least one, preferably 2 to 5 groove-like constrictions or groove-like milled portions (6) of 0.1 to 0.2 mm.

8. The fixing nail (1) according claim 1, wherein the nail head (2) is designed in a hooded manner (7).

9. The fixing nail (1) according to claim 1 wherein the tip of the nail pin (3) is designed in a harpoon-shaped manner (4).

10. The fixing nail (1) according to claim 1 wherein the tip of the nail pin (3) is designed in a lanceolate manner (5).
